(19) United States
(12) Plant Patent Application Publication
(10) Pub. No.: US 2012/0084895 P1
(43) Pub. Date: Apr. 5, 2012

(54) Ficinia plant named 'Frosty'

(50) ;

(76) Inventor: Obed Smit, Sappemeer (NL)

(21) Appl. No.: 12/924,569

(22) Filed: Sep. 30, 2010

(51) Int. Cl. A01H 5/00

(52) U.S. Cl. PLT/373

(57) ABSTRACT

A new and distinct Ficinia cultivar named ‘FROSTY’ is disclosed, characterized by distinctive white foliage with green veins and margins, and a compact, very short plant habit. The new variety is a Ficinia, normally produced as an indoor ornamental plant.

### LATIN NAME OF THE GENUS AND SPECIES

Ficinia truncata

### VARIETY DENOMINATION

‘FROSTY’

### BACKGROUND OF THE INVENTION

The new cultivar was discovered as a seedling by the inventor, Obed Smit, a citizen of the Netherlands. The parent is the unpatented variety Ficinia truncata. The new variety was discovered in 2003, by the inventor in a commercial greenhouse in Sappemeer, the Netherlands. The seedling was a result of the self crossing of the parent variety.

Asexual reproduction of the new cultivar ‘FROSTY’ by tissue culture was first performed in 2005, at a commercial laboratory in Evergem, Belgium. Multiple generations have since been produced and have shown that the unique features of this cultivar are stable and reproduced true to type on successive generations.

### SUMMARY OF THE INVENTION

The cultivar ‘FROSTY’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘FROSTY’ These characteristics in combination distinguish ‘FROSTY’ as a new and distinct Ficinia cultivar:
1. Distinctive green leaves with a white margin.
2. Linear leaves with slight undulation.

### PARENT COMPARISON

Plants of the new cultivar ‘FROSTY’ are similar to plants of the parent, Ficinia truncata in most horticultural characteristics, however, plants of the new cultivar ‘FROSTY’ are shorter. Additionally, foliage of the parent variety is light green, with green speckles and green veins, whereas ‘FROSTY’ has green foliage with a distinctive and consistent white margin.

### COMMERCIAL COMPARISON

There are a small number of varying forms of Ficinia truncata found in nature, some of which have been cultivated commercially. However, Ficinia truncata ‘Frosty’ is the only known variety of Ficinia truncata to have green foliage with a consistent white margin.

The new variety can also be compared to the unpatented Ficinia dunensis, which has also been commercialized to a small extent. Ficinia truncata ‘Frosty’ and Ficinia dunensis are similar in most horticultural characteristics, however, Ficinia dunensis has grey-green foliage which curls, whereas Ficinia truncata ‘Frosty’ has linear, slightly undulating foliage colored green with a white margin.

### BRIEF DESCRIPTION OF THE PHOTOGRAPH

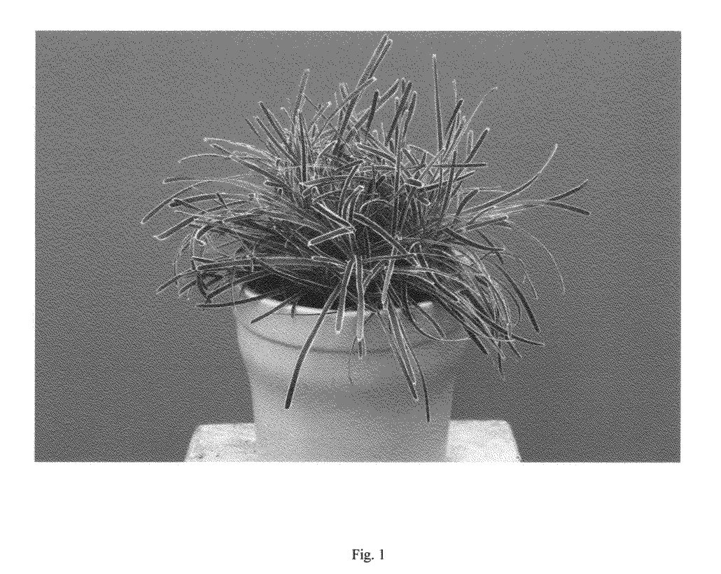

The accompanying photograph in FIG. 1 illustrates in full color a typical plant of ‘FROSTY” grown in a greenhouse, in Evergem, Belgium, in a commercial 11 cm container. Age of the plant photographed is approximately 8 months from a stage three tissue culture explant. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart except where general terms of ordinary dictionary significance are used. The following observations and measurements describe ‘FROSTY’ plants grown during the winter months in a greenhouse in Evergem, Belgium. The growing temperature ranged from 20° C. to 30° C. daytime and 18° to 20° C. at night. Measurements and numerical values represent averages of typical plant types.
Botanical classification: Ficinia truncata ‘FROSTY.’
Propagation:
Time to initiate roots: About 7 days at approximately 21-22° C.
Root description: Not very well branched, fine, coloured near RHS Brown 200C
Propagation method: Tissue culture.
Plant:
Growth habit: Flattened globular
Age of plant described: Approximately 4 months.
Container size: 10.5 cm circular
Height: Approximately 7.5 cm to top of highest leaf.
Plant spread: Approximately 16.8 cm.
Growth rate: Slow.
Branching characteristics: Basal shoots only.
Foliage:
Leaf:
   Arrangement.—Irregular basal emerging.
   Average length.—Approximately 8.1 cm.
   Average width.—Approximately 0.25 cm.
   Shape of blade.—Filiform.
   Aspect.—Slightly undulating.
   Apex.—Truncate.
   Base.—Sheathing.
   Margin.—Irregular dentate. Teeth minute.
   Texture of top surface.—Smooth, margins of a thin papery texture.
   Texture of bottom surface.—Smooth, margins of a thin papery texture.
   Quantity of leaves.—Approximately 15 leaves per tuft/shoot, approximately 40 tufts/shoots per plant approximately 600 leaves per plant.
   Color.—Young foliage upper side: Near RHS 138B, margins near RHS NN155D Young foliage under side: Near RHS 138B, margins near RHS NN155D Mature foliage upper side: Near RHS 138B, margins near RHS NN155D Mature foliage under side: Near RHS 138B, margins near RHS NN155D.
   Venation.—Type: Linear. Venation color upper side: Near RHS 137B Venation color under side: Near RHS 137B.
Flower: No flowers present, flowering not observed to date.
Other characteristics:
Seeds and fruits: Not observed to date.
Disease/pest resistance: Neither resistance nor susceptibility to pathogens and pests common to Ficinia truncata have been observed.
Temperature tolerance: The new variety tolerates temperatures' between 5 to 35° C.

What is claimed is:

1. A new and distinct cultivar of Ficinia plant named ‘FROSTY’ as herein illustrated and described.
